(19) United States
(12) Patent Application Publication
(10) Pub. No.: US 2009/0124634 A1
(43) Pub. Date: May 14, 2009

(54) Novel dipodazine compounds and applications

(75) Inventors: Lars Bohlin, Uppsala (SE); Jan Bergman, Spanga (SE); Martin Sjogren, Vastra Frolunda (SE); Ulf Goransson, Uppsala (SE)

Correspondence Address: DINSMORE & SHOHL, LLP, 1900 CHEMED CENTER, 255 EAST FIFTH STREET, CINCINNATI, OH, 45202, US

(73) Assignee: Viogard AB

(21) Appl. No.: 11/921,901

(22) PCT Filed: Jun. 9, 2006
(86) PCT No.: PCT/SE2006/050190

§ 371 Date: Dec. 10, 2007

Related U.S. Application Data
(60) Provisional application No. 60/689,479, filed on Jun. 10, 2005.

(51) Int. Cl. A61K 31/497; C07D 403/00; A61P 31/04

(52) U.S. Cl. 514/254.06; 544/373

(57) ABSTRACT

The invention relates to a novel class of dipodazine derivatives, suitable for use as surface on-growth inhibiting agents.

The present invention relates to a novel class of compounds based on the dipodazine base structure. It also relates to use of the novel compounds for inhibiting surface on-growth by various organisms.

### BACKGROUND OF THE INVENTION

The solution of the severe technical and economical problem caused by marine fouling organisms, e.g. barnacles, blue mussels, algae and hydroids, for the shipping industry and in aquaculture has been the use of TBTO (tri-n-butyl tin oxide), copper oxide and herbicides in marine coatings. However, several of these have been recognised to be toxic against non-fouling marine organisms. For example, TBTO has been ascribed effects such as reproduction failure and decrease in adult growth in oysters and the development of imposex in gastropodes such as the dog whelk. Because of these unwanted side effects, the use of TBTO will be stopped by future bans; the International Marine Organisation will recommend a global ban from the year 2008. Therefore it is urgent to find new non-toxic alternatives which exert a specific action on target organisms and which also are biodegradable.

Different bioactive substances, e.g. peptides, sterols, furanones and terpenes, have been isolated from marine organisms, mainly sessile organisms like algae, bryozoans, hydroids, tunicates and marine sponges. In several cases these secondary metabolites are thought to act as a chemical defense, in order to ward off or deter grazers, predators or larvae of fouling organisms, since these organisms lack the possibility to flee or escape such attacks. The importance of this secondary metabolite production both in terrestrial and marine organisms has been under debate for many years, but accumulating evidence favours the argument that these substances are involved in adaptive interactions between producers and target organisms and are not mere waste products.

Marine sponges are pre-eminent producers of bioactive secondary metabolites and their repertoire includes peptides, terpenes and sterols. Many of these compounds show a functional diversity of actions including antimicrobial, antiviral and cytotoxic activities. In addition, many substances shown to prevent fouling or predation have been isolated and chemically characterised from marine sponges. Bioactive compounds of sponge origin have been used as basis for the synthesis of analogues. Examples are glycolipids produced by bacteria that live associated with the marine sponge Agelas sp. and the antibacterial agelasines isolated from the marine sponge Agelas nakamurai.

### SUMMARY OF THE INVENTION

In a first aspect the present invention relates to a novel class of compounds generally defined by the following formula:

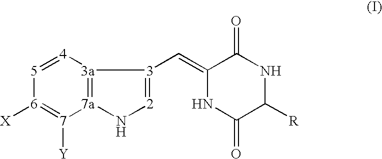

wherein

X and Y can be the same or different and can be any of H and R′, wherein
R′ is selected from —CH₃; —CH₂CH₃; —CH₃O; —Br; —Cl;
   with the proviso that X and Y cannot both be —H; or

X and Y together form a ring structure selected from

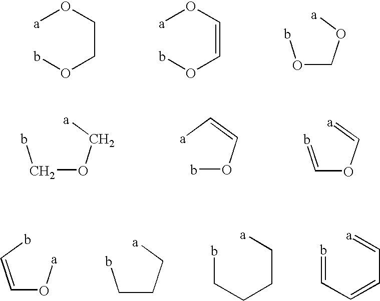

wherein a and b form points of attachment to positions 6 and 7, respectively, in formula (I);
and wherein

R is any of —H, —CH₃, —C₂H₅, C₃H₇, —CH═CH₂.

In a second aspect the invention relates to a method of inhibiting surface on-growth by organisms on various kinds of structures, comprising formulating the active compound(s) according to the invention in a suitable carrier to form a coatable composition for application onto a surface to be protected against on-growth by organisms, such as cyprids and/or mussels in sea water, or bacteria in medical environments, e.g tubings and surgical equipment, and applying said composition onto a surface to be protected

In a third aspect the invention relates to compositions for inhibiting surface on-growth by organisms on various kinds of structures. In particular such compositions are provided as any of paints, sprays, and cleaning compositions.

The paints can be based on different types of carrier media, e.g. vegetable oils, such as linseed oil, alkyd bases, or acrylic bases.

In a fourth aspect the invention relates to a method of inhibiting microfouling on the surface of surgical and other medical instruments or apparatuses, such as cutting tools for surgery, tubing for conveying body fluids such as blood and urine.

Other specific applications of the active compounds are e.g. to incorporate them in films of various polymeric materials, attachable to the surfaces to be protected. The films and active compounds are formulated such that the compounds are releasable from the film.

Also, it is possible to formulate the active compounds in vegetable oils, such as linseed oil, or soy bean oil to form pastes or lotions for application on a variety of surfaces including living organisms.

Further scope of applicability of the present invention will become apparent from the detailed description given hereinafter and the accompanying drawings which are given by way of illustration only, and thus not to be considered limiting on the present invention, and wherein

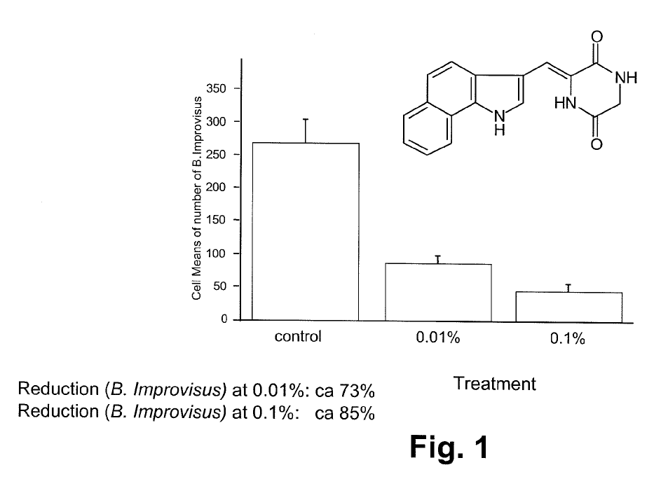

FIG. 1 shows the result of a field test with a compound according to the invention;

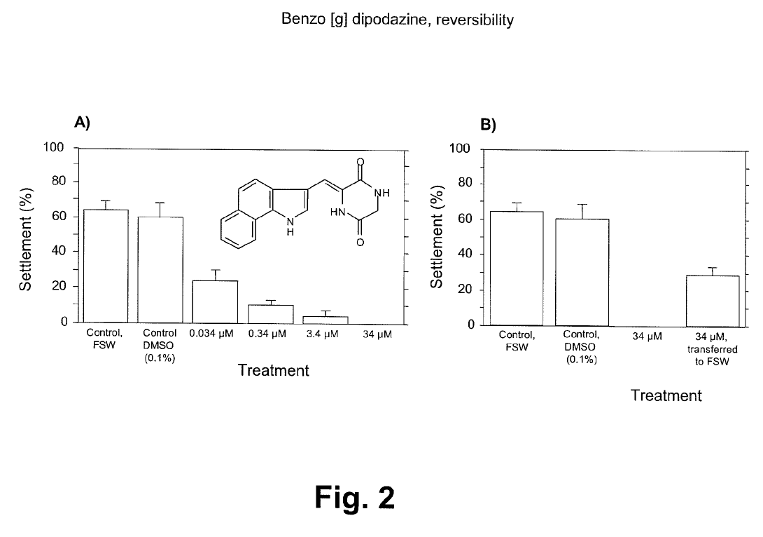

FIG. 2 is the results of a reversibility test;

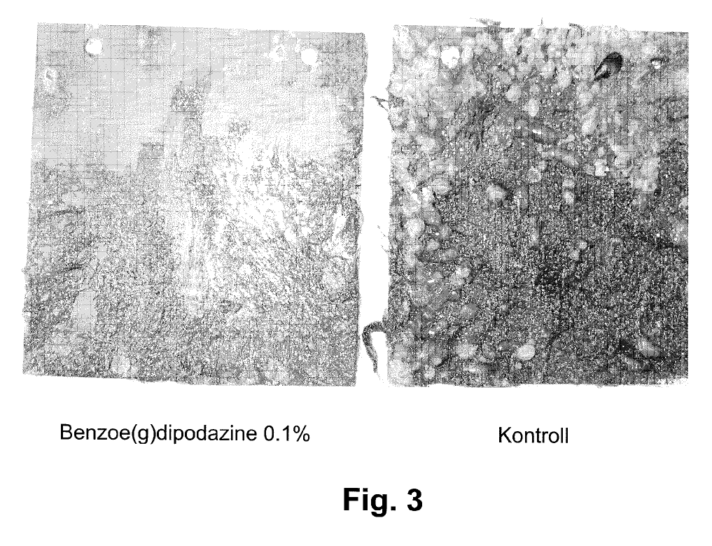

FIG. 3 are photographs of a panel treated according to the invention compared to a non-treated control panel;

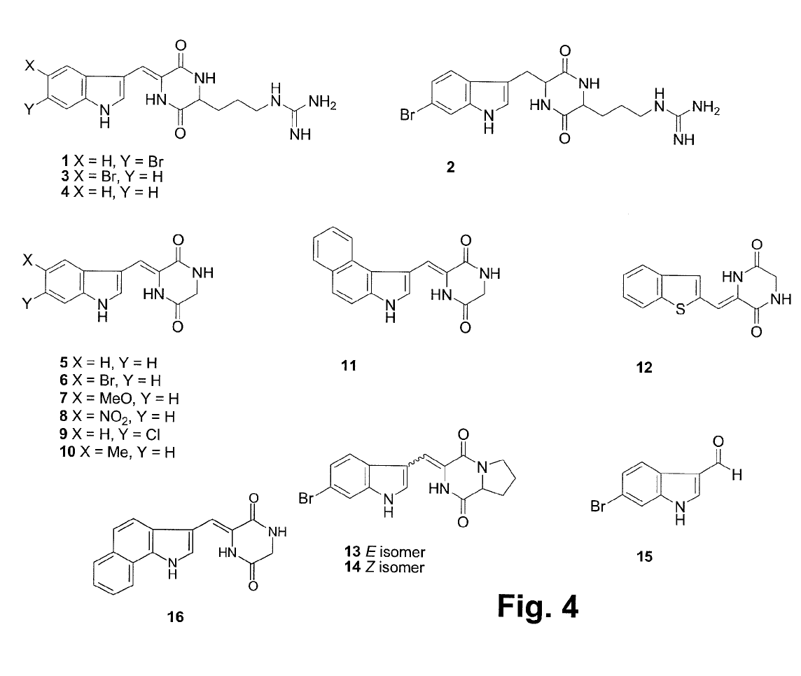

FIG. 4 shows the structure of a number of compounds i.a. barettin and analogues used for comparative purposes, and one compound according to the invention, viz. benzo[g]dipodazine.

### DETAILED DESCRIPTION OF PREFERRED EMBODIMENTS

The present invention relates in its broadest form to a novel class of compounds having an inhibiting effect on surface on-growth in a variety of environments. In particular it has utility for preventing on-growth in environments comprising water or fluids mainly constituted of water, such as sea constructions, boats, but also medical applications involving body fluids containing micro-organisms, e.g. bacteria, that might cause unwanted surface coatings.

The novel class of compounds according to the invention can suitably be used in anti-fouling products for underwater use and such products can be prepared by conventional methods.

The dipodazine and/or derivatives thereof and analogues, as defined in the claims, can for example be mixed with a binding agent such as an organopolysiloxane, e.g. a low molecular mass alkoxy-functional silicone resin, a silicone rubber or an organosilicon copolymer.

An anti-fouling composition comprising the compounds according to the invention and an organopolysiloxane can additionally comprise inorganic pigments, organic pigments, dyes (which are preferably insoluble in salt water) and/or conventional auxiliaries such as fillers, solvents, plasticizers, catalysts, inhibitors, tackifiers, coating additives and/or common dispersion auxiliaries.

Other examples of anti-fouling compositions that are meant for use under water and that can be used with the anti-fouling agents according to the present invention, are disclosed in U.S. Pat. No. 6,245,784-B1, U.S. Pat. No. 6,217,642-B1, U.S. Pat. No. 6,291,549-B1, U.S. Pat. No. 6,211,172-B1 and U.S. Pat. No. 6,172,132-B1.

The final anti-fouling products could be used i.a. for underwater structures, e.g. in plumbing ports of nuclear power stations, at ocean facilities such as bayshore roads, undersea tunnels, port facilities, and in canals or channels, machinery operated by the power of sea motion (waves), such as power plants. The agents according to the invention could also be used for coating marine vessels, fishing gear (rope, fishing net or the like).

The anti-fouling coating compositions can be applied either directly to the surface of vessel hulls and underwater structures or applied to the surface of vessel hulls and underwater structures pre-coated with undercoating material such as a rust preventive and a primer.

The anti-fouling coating compositions may also be used to repair surfaces of vessel hulls and underwater structures previously coated with a conventional anti-fouling paint or anti-fouling coating composition.

Other structures and devices that can be protected by the novel agents are exemplified by membranes, pumps and filters employed in the biotechnology process industry.

Further fields of use that are possible are the protection of medical equipment from the on-growth of bio film, i.e. bacterial and/or microbial adhesion on the surface of devices such as surgical instruments, tubing in contact with body fluids, etc.

It is also conceivable to protect fish in fish breeding plants from on-growth of unwanted species, e.g. bacteria and/or other organisms having a pathogenic effect on the fish, by the application of an agent according to the invention, either directly on the body of the animal, or by administration in the water or with the fodder. Possibly, also other animals such as cattle could be protected from infestation or attacks by vermins.

For the purpose of the present invention, we have used barettin (1) (see FIG. 4 for structures of compounds 1-16 below) and dipodazine (5) as the basis for the synthesis of 14 analogues. Barettin (1) was isolated from the marine sponge Geodia barretti Bowerbank (family Geodiidae, class Demospongiae, order Astrophorida), chemically characterised [27, 28] and synthesised [14] in our previous work. In addition to barettin, we also reported 8,9-dihydrobarettin (2) in that work. Barettin (1) and 8,9-dihydrobarettin (2) belong to the substance class of diketopiperazines (DKP:s) which have attracted attention as a group of compounds with a number of different bioactivities. The two compounds (1 and 2) were isolated guided by their ability to inhibit settlement of settling stage larvae (cypris larvae) of the barnacle Balanus improvisus Darwin (Cirripedia, Crustacea). The bioactivity differed an order of magnitude between barettin (EC₅₀-0.9 μm) and 8,9-dihydrobarettin (EC₅₀-7.9 μM) and none of the compounds displayed any significant effect on larval mortality [27]. Similar differences in activity were observed when the baretttins were tested for their antifouling effect on settlement of B. improvisus and the blue mussel Mytilus edulis larvae in a field experiment. Dipodazine (5) that has been used as the basis for a number of the analogues in this study is also a DKP. While dipodazine shares the tryptophan moiety with barettin, the arginine is replaced by a glycine residue.

The main goals for the synthesis and subsequent tests for settlement bioactivity of the analogues presented in the present work are to gain an increased knowledge into the structure-activity basis of bioactivity and to design compounds with increased antifouling effect along with preserved non-toxic effect.

### MATERIALS AND METHODS

### 1. Peptide Synthesis

Barettin (1) was used as a starting template for the design of two analogues, namely 5-bromobarettin (3) and debromobarettin (4) (FIG. 1). Dipodazine (5) was used in the present study as starting template for the remaining 12 analogues: 5-bromodipodazine (6), 5-methoxydipodazine (7), 5-nitrodipodazine (8), 6-chlorodipodazine (9), 5-methyldipodazine (10), benzo[e]dipodazine (11), 3-[1-benzothiophen-2-yl-methylidene]-piperazine-2,5-dione (12), 3-[1-(6-bromo-1H-indol-3-yl)-meth-(E)-ylidene]-hexahydro-pyrrolo[1,2-a]pyrazine-1,4-dione (13), 3-[1-(6-bromo-1H-indol-3-yl)-meth-(2)-ylidene]-hexahydro-pyrrolo[1,2-a]pyrazine-1,4-dione (14), 6-bromo-1H-indole-3-carboxaldehyde (15) and benzo[g]dipodazine (16).

NMR spectra were recorded at 300 MHz for ¹H and 75 MHz for ¹³C, respectively. NMR spectra were recorded in DMSO-d₆, using the solvent signal as reference. δ values are given in ppm, coupling constants are given in Hz. The IR spectra were acquired using a FT-IR instrument. Melting points were determined using the capillary method and are uncorrected. All reagents used were purchased from Aldrich, Lancaster, Merck or Biosynth and were used as received.

For MS a nanospray-ion trap MS [Protana's NanoES source (MDS Protana A/S, Odense, Denmark) mounted on a LCQ (Thermo Finnigan, San Jose, Calif.)] was used. Samples were analyzed in the positive ion mode, directly after fractionation, or were lyophilized and dissolved in 60% MeOH with 1% HOAc. The spray voltage was set to 0.5 kV and the capillary temperature to 150° C. Barettin (1), 8,9-dihydrobarettin (2), dipodazine (5), 3-[1-(6-bromo-1H-indol-3-yl)-meth-(E/Z-ylidene]-hexahydro-pyrrolo[1,2-a]pyrazine-1,4-dione (13, 14) and 6-bromo-1H-indole-3-carboxaldehyde (15) were prepared as reported in literature. 5-bromobarettin (3) and debromobarettin (4) were synthesised using the same method as for the preparation of barettin (1). The dipodazine analogues (6-12, 16) were prepared as dipodazine (in some cases potassium-tert-butoxide were used instead of caesium carbonate as the base, which worked equally well).

### 2. Larval Settlement Assay

Barnacle cyprids larvae of B. improvisus was reared in the laboratory as described in [2]. The settlement propensity and mortality of barnacle cyprids were used to evaluate the bioactive effect of barettin and analogues. Stock solutions of all compounds shown in FIG. 4 were prepared by dissolving the compounds in dimethylsulfoxide (DMSO). The stock solutions were further diluted in filtered seawater (FSW, Millipore 0.2 μm) to yield the desired concentrations of 1×10⁻⁴, 1×10⁻³ and 1×10⁻² mg ml⁻¹. The settlement assays were performed using Petri dishes of untreated polystyrene (Nunc no 240045, Ø 48 mm) containing 10 ml of FSW to which 20±5 cyprids were added. Cyprids were used on their first or second day after moulting. Each treatment with a specific analogue was replicated 4 times and dishes with FSW only or FSW containing DMSO (0.1%) served as controls. Dishes were maintained for 3-4 days at room temperature (21±2° C.) in the prevailing light:dark cycle of 9:15 h. The effects of the different analogues, classified as either neutral, stimulatory or inhibitory, on the cyprids, were determined by examining the dishes under a stereo microscope and checking for 1) attached and metamorphosed individuals, 2) non-metamorphosed, alive, non-attached cyprids and 3) dead cyprids. Each substance was tested at least in two independent experimental series.

Data for verification of the identity of the synthesized compounds are given below:

### 5-bromobarettin (3)

Yield: 83%; Mp 144-146° C.; IR (neat): 3166, 1655, 1613, 1433, 1391, 1232, 746 cm-1; ¹H NMR (DMSO-d₆) δ 11.96 (s, 1H), 9.65 (s, 1H), 8.41-8.40 (m, 1H), 7.97 (d, 1H), 7.78-7.77 (m, 2H), 7.41 (m, 1H), 7.78-7.27 (br, 3H), 7.27 (m, 1H), 6.94 (s, 1H), 4.03-4.01 (m, 1H), 3.21-3.14 (m, 2H), 1.76-1.67 (m, 2H), 1.55-1.45 (m, 2H); ¹³C NMR (DMSO-d₆) δ 166.7 (s), 160.8 (s), 157.0 (s), 134.4 (s), 128.8 (s), 127.9 (d), 124.5 (d), 123.0 (s), 120.6 (d), 113.9 (d), 112.5 (s), 107.6 (s), 107.1 (s), 54.6 (d), 41.1 (t), 31.2 (t), 24.1 (t). MS (ESI): m/z 419.1 (M+H)⁺.

### debromobarettin (4)

Yield: 63%; Mp: 242-244° C.; IR (neat): 3160, 1650, 1618, 1433, 1233, 744 cm⁻¹; ¹H NMR (DMSO-d₆) δ 11.80 (s, 1H), 9.54 (s, 1H), 8.37 (d, J=2.3, 1H), 7.94 (d, J=2.8, 1H), 7.87 (t, J=6.0, 1H), 7.64-7.01 (br, 3H), 7.64 (d, J=7.8, 1H), 7.44 (d, J=8.3, 1H), 7.18-7.09 (m, 2H), 7.09 (s, 1H), 4.05-4.01 (m, 1H), 3.14-3.11 (m, 2H), 1.76-1.74 (m, 2H), 1.56-1.53 (m, 2H); ¹³C NMR (DMSO-d₆) δ 166.8 (s), 161.0 (s), 157.1 (s), 135.7 (s), 127.0 (s), 126.5 (d), 122.3 (s), 122.1 (d), 119.9 (d), 118.1 (d), 111.9 (d), 107.9 (s), 107.8 (s), 54.7 (d), 40.2 (t), 31.2 (t), 24.1 (t). MS (ESI): m/z 341.1 (M+H)⁺.

### 5-bromodipodazine (6)

Yield: 98%; Mp: 305-307° C.; IR (neat): 3198, 1672, 1619, 1443, 1392, 1231, 796, 745 cm⁻¹; ¹H NMR (DMSO-d₆) δ 11.81 (s, 1H), 9.56 (s, 1H), 8.15 (s, 1H), 7.97 (d, J=2.6, 1H), 7.79 (d, J=1.7, 1H), 7.42 (d, J=8.6, 1H), 7.30 (dd, J=1.9, 8.6, 1H), 6.93 (s, 1H), 4.01 (s, 2H); ¹³C NMR (DMSO-d₆) δ 164.5 (s), 160.5 (s), 134.4 (s), 128.8 (s), 127.6 (d), 124.5 (d), 123.3 (s), 120.6 (d), 113.8 (d), 112.4 (s), 107.6 (s), 106.7 (d), 44.9 (t). MS (ESI): m/z 320.1 (M+H)⁺.

### 5-methoxydipodazine (7)

Yield: 43%; Mp: 296-299° C.; IR (neat): 3255, 3042, 2933, 1673, 1624, 1435, 1389, 1337, 1213, 821 cm⁻¹; ¹H NMR (DMSO-d₆) δ 11.50 (s, 1H), 9.42 (s, 1H), 8.01 (s, 1H), 7.88 (d, J=2.7, 1H), 7.33 (d, J=8.8, 1H), 7.10 (d, J=2.3, 1H), 6.98 (s, 1H), 6.82 (dd, J=2.3, 8.8, 1H), 4.00 (s, 2H), 3.80 (s, 3H); ¹³C NMR (DMSO-d₆) δ 164.4 (s), 160.9 (s), 154.1 (s), 130.6 (s), 127.5 (s), 126.7 (d), 122.2 (s), 112.6 (d), 112.4 (d), 108.0 (d), 107.8 (s), 99.7 (d), 55.3 (q), 44.9 (t). MS (ESI): m/z 272.1 (M+H)⁺.

### 5-nitrodipodazine (8)

Yield: 70%; Mp: 310-312° C. IR (neat): 3400, 3196, 3114, 1690, 1628, 1515, 1437, 1411, 1322, 1096, 737 cm⁻¹; ¹H NMR (DMSO-d₆) δ 12.27 (s, 1H), 9.72 (s, 1H), 8.57 (d, J=2.0, 1H), 8.23 (s, 1H), 8.15 (d, J=1.2, 1H), 8.08 (dd, J=2.1, 8.9, 1H), 7.63 (d, J=8.9, 1H), 6.99 (s, 1H), 4.03 (s, 2H); ¹³C NMR (DMSO-d₆) δ 164.5 (s), 160.2 (s), 141.1 (s), 138.8 (s), 129.6 (d), 126.4 (s), 124.6 (s), 117.3 (d), 115.4 (d), 112.4 (d), 110.3 (s), 105.5 (d), 44.9 (t).

MS (ESI): m/z 287.1 (M+H)⁺.

### 6-chlorodipodazine (9)

Yield: 58%; Mp: 344-345° C.; IR (KBr): 3136, 3042, 2926, 1693, 1661, 1445, 1408, 1233, 1166, 1097, 837, 800 cm⁻¹; ¹H NMR (DMSO-d₆) δ 11.73 (s, 1H), 9.54 (s, 1H), 8.14 (s, 1H), 7.96 (d, J=1.8, 1H), 7.66 (d, J=8.5, 1H), 7.47 (d, J=1.4, 1H), 7.13-7.10 (dd, J=1.6, 8.5, 1H), 6.95 (s, 1H), 4.01 (s, 2H); ¹³C NMR (DMSO-d₆) δ 164.4 (s), 160.5 (s), 136.0 (s), 127.1 (d), 126.6 (s) 125.7 (s), 123.3 (s), 120.0 (d), 119.6 (d), 111.3 (d), 108.0 (s), 106.7 (d), 44.8 (t). MS (ESI): m/z 276.1 (M+H)⁺.

### 5-methyldipodazine (10)

Yield: 33%; Mp: 301-302° C.; IR (KBr): 3389, 3194, 3054, 1691, 1620, 1484, 1449, 1398, 1350, 1307, 1233, 1198, 794, 598, 449 cm⁻¹; ¹H NMR (DMSO-d₆) δ 11.51 (s, 1H), 9.43 (s, 1H), 8.09 (s, 1H), 7.89 (d, J=1.6, 1H), 7.43 (s, 1H), 7.32 (d, J=8.2, 1H), 7.00 (s, 1H), 6.99 (s, 1H), 4.00 (s, 2H) 2.40 (s, 3H); ¹³C NMR (DMSO-d₆) δ 164.3 (s), 160.7 (s), 133.8 (s), 128.5 (s), 127.2 (s), 126.1 (d), 123.6 (d), 122.1 (s), 117.6 (d), 111.4 (d), 107.6 (d), 107.3 (s), 44.8 (t), 21.2 (q). MS (ESI): m/z 256.2 (M+H)⁺.

### benzo[e]dipodazine (11)

Yield: 61%; Mp: 284° C.; IR (KBr): 3315, 3036, 1681, 1660, 1618, 1445, 1380, 1227, 1174, 1093, 994, 800, 748, 693 cm⁻¹; ¹H NMR (DMSO-d₆) δ 11.96 (s, 1H), 9.59 (s, 1H), 8.35 (s, 1H), 8.32 (d, J=4.4, 1H), 7.97 (d, J=7.9, 1H), 7.74 (d, J=2.0, 1H), 7.62-7.54 (m, 3H), 7.43-7.35 (m, 2H), 4.04 (s, 2H); ¹³C NMR (DMSO-d₆) δ 164.0 (s), 160.1 (s), 133.0 (s), 129.9 (d), 128.8 (d), 128.5 (s), 125.8 (d), 124.7 (s), 124.2 (d), 122.9 (d), 122.5 (d), 119.4 (s), 113.7 (d), 109.8 (s), 109.4 (d), 44.8 (t). MS (ESI): m/z 292.2 (M+H)⁺.

### 3-[1-benzothiophen-2-yl-methylidene]-piperazine-2,5-dione (12)

Yield: 83%; Mp: 258° C.; IR (KBr): 3211, 3052, 1680, 1625, 1424, 1341, 1206, 1165, 1079, 812.1, 748.1 cm⁻¹; ¹H NMR (DMSO-d₆) δ 9.80 (s, 1H), 8.39 (s, 1H), 7.99-7.96 (m, 1H), 7.86-7.83 (m, 1H), 7.73 (s, 1H), 7.42-7.34 (m, 2H), 6.88 (s, 1H), 4.06 (s, 2H); ¹³C NMR (DMSO-d₆) δ 164.6 (s), 159.4 (s), 139.5 (s), 139.2 (s), 135.8 (s), 127.6 (s), 125.4 (d), 125.0 (d), 124.8 (d), 122.2 (d), 107.1 (d), 44.77 (t). MS (ESI): m/z 259.1 (M+H)⁺.

### benzo[g]dipodazine (16)

Yield: (71%); Mp: 356° C. IR (KBr): 3196, 1654, 1609, 1386, 1215, 1206, 843, 750, 554 cm⁻¹. ¹H NMR (DMSO-d₆) δ 12.53 (s, 1H), 9.56 (s, 1H), 8.40 (d, J=8.1, 1H), 8.17 (s, 1H), 8.03 (s, 1H), 7.97 (d, J=8.2, 1H), 7.79 (d, J=8.7, 1H), 7.60-742 (m, 2H), 7.09 (s, 1H), 4.03 (s, 2H). ¹³C NMR (DMSO-d₆) δ 164.4 (s), 160.6 (s), 130.3 (s), 130.0 (s), 128.3 (d), 125.6 (d), 124.0 (d), 123.9 (d), 123.0 (s), 122.8 (s), 121.8 (s), 120.6 (d), 120.5 (d), 118.2 (d), 44.9 (t). MS (ESI): m/z 292.2 (M+H)⁺.

The compound benzo[g]dipodazine (16) (or 1-H-Benzo[g]indole-dipodazine) was synthesized using the route shown below:

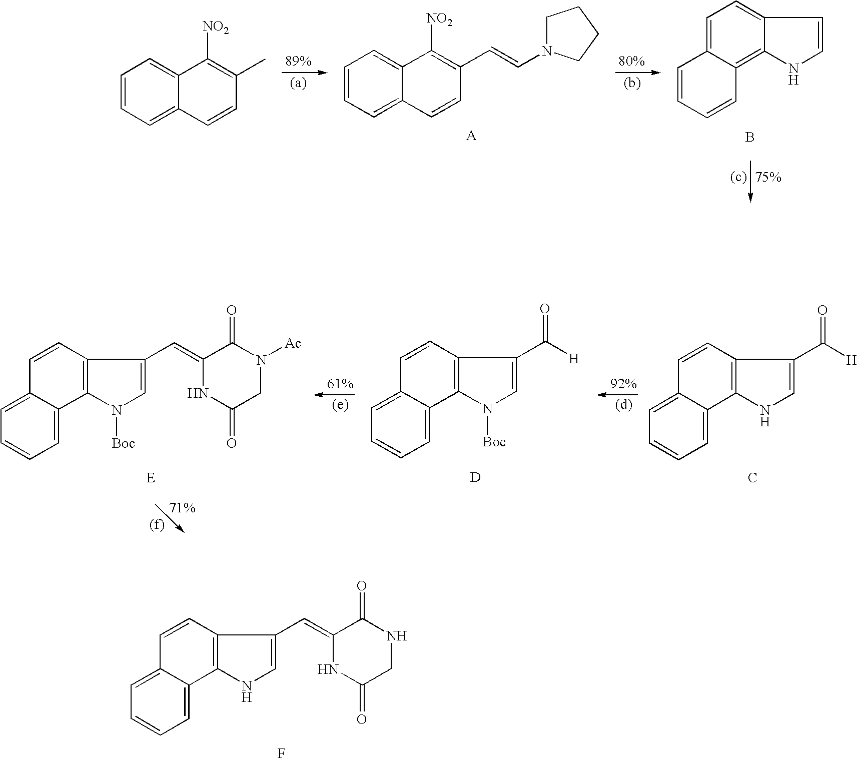

Scheme 1. (a) i. DMFDMA, ˜110° C., DMF ii. Pyrrolidine, 3 h. (b) EtOH/CH₂Cl₂, Raney-Ni/H₂, ˜3 h. (c) i. POCl₃, DMF, 80-90° C. 1 h. ii. aq NaOH (50%), reflux 2 min, H₂O (d) i. THF, DMAP, Boc, Room temperature. (e) t-BuOK, t-BuOH/DMF, 0° C. to room temperature, 18 h. (f) i. EtOH, KOH, 80-85° C., 0.25 h ii. HCl.

### Compound (A)

N,N-dimethylformamide dimethyl acetal (2.75 g, 23 mmol) and pyrrolidine (1.64 g, 23 mmol) was added to a solution of nitronaphtalin (3.75 g, 20 mmol) in DMF (11 ml). The solution was refluxed (110° C.) for 3 hr under nitrogen gas and allowed to cool in room temperature. The volatile components were evaporated and the residue was dissolved in dichloromethane (6 mL) and methanol (48 mL). The solution was concentrated to a volume of 46 mL on a steam bath and cooled to SOC. Filtration and washing of the product with cold methanol afforded 4.2 g of dark red crystals. The mother liquor was evaporated and the residue was recrystallized from methanol to give an additional 0.6 g of red solid. Yield: 4.8 g (89%). Data in agreement with the literature.

### 1-H-Benzo[g]indole (B)

Compound A (6 g, 22.4 mmol) was dissolved in methanol (15 mL) and dichloromethane (30 mL). Hydrogenation in presence of Raney-Ni (3 hr) gave an indole derivative as an orange solid. Yield: 3 g (80%); mp:

¹H NMR (DMSO-d₆) δ 11.99 (s, 1H), 8.37 (d, 1H, J=8.2 Hz), 7.92 (d, 1H, J=8.0 Hz), 7.7 (d, 1H, J=8.6 Hz), 7.54 (app t, 1H, J=7.5 Hz), 7.45-7-36 (m, 3H), 6.59-6.57 (m, 1H).

### 1-H-Benzo[g]indole-3-carboxaldehyde (C)

POCl₃ (0.6 μg, 4.0 mmol) was added dropwise to DMF (2 mL) at 0° C. When the addition was complete a solution of Benzo[g]indole (B) (0.6 g, 3.6 mmol) in DMF (0.7 mL) was added dropwise to the stirred mixture. The mixture was heated at 80-90° C. for 1 h. Ice (ca 4 g) was added to the reaction mixture and NaOH (3 mL, 50%) was slowly added. The solution was heated to reflux for 2 min and set aside to cool. The resulting precipitate was collected to afford compound C as a light red solid. Yield: 0.530 g (75%).

¹H NMR (DMSO-d₆) δ 13.00 (s, 1H), 10.04 (s, 1H), 8.44 (d, 1H, J=8.2 Hz), 8.37 (s, 1H), 8.21 (d, 1H, J=8.6 Hz), 8.01 (d, 1H, J=8.1 Hz), 7.70-7-59 (m, 2H), 7.54 (app t, 1H, J=7.5 Hz).

### 1-H-Benzo[g]-(N-tert.-butyl carbonato)-indole-3-carboxaldehyde (D)

Compound C (1.0 g, 5.13 mmol) and Di-tertbutyl carbonate (1.7 g, 7.7 mmol) were dissolved in dry THF (15 mL) in room temperature under N₂ gas and 4-dimethylaminopyrridine (0.08 g, catalysis) was added. A precipitate was formed which was quenched with water. The resulting solid was filtrated and recrystallized from ethanol. Yield: 1.4 g (92%); mp ° C.

¹H NMR (DMSO-d₆) δ 10.04 (s, 1H), 8.76 (s, 1H), 8.62 (d, 1H, J=8.5 Hz), 8.29 (d, 1H, J=8.6 Hz), 8.07 (d, 1H, J=8.4 Hz), 7.93 (d, 1H, J=8.6 Hz), 7.65-7-53 (m, 2H).

### 1-Acetyl-3-[1-H-benzo[g]-[(N-tert.-butyl carbonato)-indole]-meth-(Z)-ylidene]-piperazine-2,5-dione (E)

Compound D (1.0 g, 3.4 mmol) and 1,4-diacetyl-piperazine-2,5-dione (1.35 g, 6.8 mmol)) were dissolved in dry DMF (10 mL) at 0° C. t-BuOK (0.5 g, 4 mmol) in t-BuOH/DMF (12/10 mL) was added dropwise to the reaction mixture and was allowed to reach room temperature. The mixture was stirred for about 18 h and then poured into ice water (15 mL). A yellow precipitate was collected and washed with absolute ethanol. Yield: 0.9 g (61%); mp: ° C.

¹H NMR (DMSO-d₆) δ 10.42 (s, 1H), 8.60 (d, 1H, J=8.4 Hz), 8.2 (s, 1H), 8.05 (d, 1H, J=7.7 Hz), 7.85 (s, 1H), 7.62-7-50 (m, 2H), 7.22 (s, 1H), 4.40 (s, 2H), 2.54 (s, 3H), 1.70 (s, 9H).

### 1-H-Benzo[g]indole-dipodazine (F)

A solution of compound E (0.2 g, 0.46 mmol) and 1 M KOH/EtOH (8/10 mL) was stirred at 80-85° C. for 25 min, thereafter EtOH was evaporated by reduced pressure. The reaction mixture was acidified with 2 M HCl to pH 4 and H₂O (10 mL) was added. The precipitate was collected and washed with ethanol to give compound F (i.e. the same compound as referred to as (16) in the discussion above) as an off-white solid. Yield: (71%); mp: 356° C.

IR (KBr): 3196, 1654, 1609, 1386, 1215, 1206, 843, 750, 554 cm⁻¹. ¹H NMR (DMSO-d₆) δ 12.53 (s, 1H),), 9.56 (s, 1H), 8.40 (d, 1H, J=8.1 Hz), 8.17 (s, 1H), 8.03 (s, 1H), 7.97 (d, 1H, J=8.2 Hz), 7.79 (d, 1H, J=8.7 Hz), 7.60-742 (m, 2H), 7.09 (s, 1H), 4.03 (s, 2H). ¹³C NMR (DMSO-d₆) δ 164.4 (s), 160.6 (s), 130.3 (s), 130.0 (s), 128.3 (d), 125.6 (d), 124.0 (d), 123.9 (d), 123.0 (s), 122.8 (s), 121.8 (s), 120.6 (d), 120.5 (d), 118.2 (d), 44.9 (t).

Without wishing to be bound by any theory, the inventors assume that the structural feature responsible for the surprising effect of the compounds according to the invention, is the space filling feature of the substituent(s) (such as a benzene ring) in the 6,7-position of the dipodazine base structure. Probably this structural element fits in and blocks an essential structural element of a protein on the surface of organisms which are prone to adhere to and grow on surfaces in general, and which is responsible for the binding. Therefore, it is very likely that other structural elements having a similar three-dimensional structure will exhibit similar effect.

It is therefore suggested that the class of compounds according to the invention be defined by the following general formula:

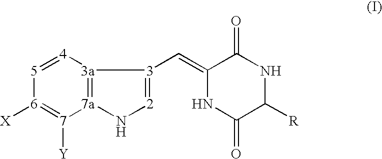

wherein

X and Y can be the same or different and can be any of H and R′, wherein
R′ is selected from —CH₃; —CH₂CH₃; —CH₃O; —Br; —Cl;
   with the proviso that X and Y cannot both be —H; or

X and Y together form a ring structure selected from

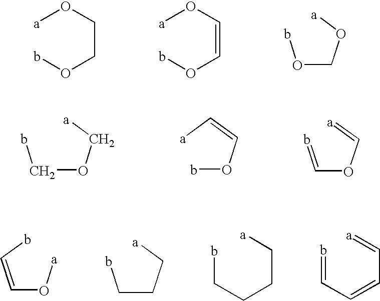

wherein a and b form points of attachment to positions 6 and 7, respectively, in formula (I);
and wherein

R is any of —H, —CH₃, —C₂H₅, C₃H₇, —CH═CH₂.

The at present most preferred compound according to the invention is benzo[g]dipodazine according to the formula:

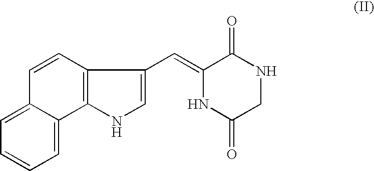

The novel compounds according to the invention are usable in general for preventing on-growth by various kinds of organisms on surfaces. In particular they can be used for protective coatings on under water structures, be it boats or ships or stationary structures such as concrete constructions in harbours etc.

As already mentioned the novel compounds are particularly suited for protection from on-growth by cyprids on e.g. boat hulls. Suitably, the active compound(s) are formulated as a component in a paint, by techniques known in the art of paint making.

Below the use of benzo[g]dipodazine as on-growth inhibitor was studied in field studies, and results of such studies are discussed below by way of Examples.

### EXAMPLES

### Example 1

### Comparative Field Experiment with Barettin

The effects of barettin and 8,9-Dihydrobarettin on B. improvisus recruitment was studied, by formulating different paints with different concentrations of the active substances.

The paints were commercially available paints and the four different paints are listed below:

| Paint name | Paint technology | Paint manufacturer |
|---|---|---|
| SPF | Self-polishing paint (SPC) | Lotréc, Lidingö, Sweden |
| FabiEco | SPC | International, Newcastle, UK |
| TF | Solid paint/weak SPC | Lotréc, Lidingö, Sweden |
| H2000 | Solid paint/weak SPC | Lotréc, Lidingö, Sweden |

There was a significant reduction of the recruitment of B. improvisus in all treatments of barettin and 8,9-dihydrobarettin in the SPF paint as compared to control panels, except when using 8,9-dihydrobarettin in the concentration 0.01% (summarized in Table 1). When using barettin in SPF at the concentrations 0.1% and 0.01%, the recruitment of B. improvisus was reduced by 89% and 67%, respectively (Table 1). When 8,9-dihydrobarettin was used in SPF, the recruitment of barnacles was reduced by 61% in the concentration of 0.1% (Table 1) and by 43% in the concentration 0.01%.

TABLE 1

| | barettin | | | barettin | | | 8,9-dihydro-barettin | | | 8,9-dihydro-barettin | | |
|---|---|---|---|---|---|---|---|---|---|---|---|---|
| | (0.1%) | | | (0.01%) | | | (0.1%) | | | (0.01%) | | |
| | Reduction of | F- | | Reduction of | F- | | Reduction of | F- | | Reduction of | F- | |
| | recruitment | value | p- | recruitment | value | p- | recruitment | value | p- | recruitment | Value | p- |
| Paint | (%) | (F_{1, 60}) | value | (%) | (F_{1, 60}) | value | (%) | (F_{1, 60}) | value | (%) | (F_{1, 60}) | value |

(a) (B. improvisus)

| | | | | | | | | | | | | |
|---|---|---|---|---|---|---|---|---|---|---|---|---|
| SPF | 89*** | 49.2 | 0.0001 | 67** | 12 | 0.001 | 61** | 7.5 | 0.008 | 43 | 0.6 | 0.4 |
| FabiEco | 49 | 3.7 | 0.06 | 45 | 3.1 | 0.08 | 69*** | 14.5 | 0.0003 | 59* | 7.3 | 0.009 |
| TF | 17 | 0.9 | 0.4 | 25 | 0.9 | 0.4 | −1 | 0.03 | 0.9 | −10 | >0.0001 | 1.0 |
| H2000 | 23 | 0.7 | 0.4 | 28 | 0.8 | 0.4 | 20 | 0.5 | 0.5 | 46 | 3.2 | 0.3 |

(b) M. edulis

| | | | | | | | | | | | | |
|---|---|---|---|---|---|---|---|---|---|---|---|---|
| SPF | 81*** | 19.8 | 0.0001 | 83** | 17.8 | 0.0001 | 72** | 8.3 | 0.006 | 52 | 3.1 | 0.08 |
| FabiEco | 47 | 2.2 | 0.14 | 51 | 3.1 | 0.08 | 67** | 10.1 | 0.002 | 50* | 4.3 | 0.04 |
| TF | 6 | 0.004 | 0.9 | 51* | 5.1 | 0.03 | 3.8 | >0.001 | 1.9 | −28 | 0.5 | 0.5 |
| H2000 | 12 | 0.08 | 0.8 | 26 | 0.5 | 0.5 | 39 | 1.2 | 0.3 | 53* | 4.8 | 0.03 |

For the FabiEco paint, the recruitment of B. improvisus was reduced by 49% with 0.1% barettin in the paint and by 45%, with 0.01% barettin. This reduction was not significant as compared to the colonisation of the control panels (Table 1). On the panels coated with FabiEco containing 0.1% and 0.01% 8,9-dihydrobarettin, the recruitment of B. improvisus was reduced by 69% and 59%, in the concentrations 0.1% and 0.01%, respectively. The colonisation of B. improvisus in respect to both of the treatments with 8,9-dihydrobarettin was significantly different from the colonisation on the control panels (Table 1). There were no significant effects on the recruitment of B. improvisus in the treatments using barettin and 8,9-dihydrobarettin in the TF or H2000 paints (Table 1).

Also, the effects of barettin and 8,9-dihydrobarettin on M. edulis recruitment were studied. When analyzing the data on the recruitment of M. edulis in the present field experiment, in a 3-factor analysis of variance with substance, paint and concentration as fixed factors, there was a significant interaction between the factors substance and paint. The main contribution to the significant interaction between the factors substance and paint is the result of barettin in combination with the SPF paint. This result is significantly different from all other combinations of substance and paint, except for the combination of 8,9-dihydrobarettin in the SPF paint (SNK-test). The analysis also revealed a significant interaction between the factors concentration and substance. However, the SNK-test could not reveal any significant differences between data.

Both concentrations of barettin in the paint SPF significantly reduced the recruitment of M. edulis over an 8-week period as compared to the control panels painted with SPF without the addition of the active compound. In the SPF paint, barettin reduced the recruitment of M. edulis by 81% and 83% in 0.1 and 0.01% treatments, respectively (Table 1) as compared to the control panels. SPF with 8,9-dihydrobarettin included, reduced recruitment by 72% and 52% in 0.1% and 0.01%, respectively (Table 1). However, only the reduction in M. edulis recruitment for the treatment with 0.1% 8,9-dihydrobarettin was significantly different from control panels (Table 1).

Panels coated with the FabiEco paint to which barettin had been added, did not significantly reduce recruitment of M. edulis as compared to controls (Table 1). However, the treatments with 8,9-dihydrobarettin included in FabiEco reduced recruitment of M. edulis with 67% and 50%, in the concentrations 0.1% and 0.01%, respectively (Table 1). There were no significant effects on the recruitment of M. edulis for the treatments with barettin and 8,9-dihydrobarettin included in the TF or the H2000 paints (Table 1), except for two treatments. Barettin included in the TF-paint in the concentration 0.01% reduced recruitment of M. edulis by 51% as compared to the control panels (Table 1). Also, 8,9-dihydrobarettin included in the concentration 0.01% in the H2000-paint reduced recruitment of M. edulis by 53% as compared to the control panels (Table 1).

### Example 2

### Field Experiment with benzo[g]dipodazine

One compound according to the present invention, benzo[g]dipodazine, which is the at present preferred compound, was formulated in a commercially available paint (SPF, from Lotréc, Lidingö, Sweden). The concentration of benzo[g]dipodazine in the paint was 0.1% and 0.01% based on the weight of the paint.

The paint was coated on panels in the same way as described above in Example 1 (comparative example). A control panel was coated with the same paint without benzo[g]dipodazine. All panels were immersed in sea water at a depth of 0.2-1.5 m, and maintained in the water for eight weeks.

FIG. 1 shows the result of the test, and a clear effect can be seen, i.e. the reduction of settlement of B. Improvisus at the concentration of 0.01% was about 73%, and at 0.1% the reduction was about 85%.

FIG. 2 shows a reversibility test, i.e. the ability of cyprids to settle again in fresh seawater after having been exposed to benzo[g]dipodazine in dishes. In A) the inhibition of settlement at different concentrations of active compound is shown. At 34 μM a complete inhibition can be seen. In B) it is clearly shown that the cyprids that were totally inhibited against settlement, regained this capability when transferred to fresh sea water (FSW).

Furthermore, visual inspection of test panels revealed that the panels having been coated with a paint containing the compound according to the invention exhibit a significantly lower degree of surface on-growth than the control panels (see FIG. 3).

### REFERENCES

### References

[1] Bakkestuen A K, Gundersen L L, Petersen D, Utenova B T, Vik A. Synthesis and antimycobacterial activity of agelasine E and analogues. Org. Biomol. Chem. 2005; 3:1025-33.
[2] Berntsson K M, Jonsson P R, Lejhall M, Gatenholm P. Analysis of behavioural rejection of micro-textured surfaces and implications for recruitment by the barnacle Balanus improvisus. J. Exp. Mar. Biol. Ecol. 2000; 251:59-83.
[3] Capon R J, Ford J, Lacey E, Gill J H, Heiland K, Friedel T. Phoriospongin a and b: Two new nematocidal depsipeptides from the australian marine sponges Phoriospongia sp and Callyspongia bilamellata. J. Nat. Prod. 2002; 65:358-63.
[4] de Nys R, Dworjanyn S A, Steinberg P D. A new method for determining surface concentrations of marine natural products on seaweeds. Mar. Ecol.-Prog. Ser. 1998; 162:79-87.
[5] de Nys R, Wright A D, König G M, Sticher O. New halogenated furanones from the marine alga Delisea-pulchra (cf fimbriata). Tetrahedron 1993; 49:11213-20.
[6] De Rosa S, Mitova M, Tommonaro G. Marine bacteria associated with sponge as source of cyclic peptides. Biomol. Eng. 2003; 20:311-6.
[7] England L J, Imperial J, Jacobsen R, Craig A G, Gulyas J, Akhtar M, Rivier J, Julius D, Olivera B M. Inactivation of a serotonin-gated ion channel by a polypeptide toxin from marine snails. Science 1998; 281:575-8.
[8] Faulkner D J. Marine natural products. Nat. Prod. Rep. 2002; 19:1-48.
[9] Firn R D, Jones C G. The evolution of secondary metabolism—a unifying model. Mol. Microbiol. 2000; 37:989-94.
[10] Gauvin A, Smadja J, Aknin P A, Gaydou E M. Cyclopropane-containing sterols in the marine sponge Petrosia spheroida. Comp. Biochem. Physiol. B-Biochem. Mol. Biol. 1998; 121:451-6.
[11] Hirota H, Okino T, Yoshimura E, Fusetani N. Five new antifouling sesquiterpenes from two marine sponges of the genus axinyssa and the nudibranch Phyllidia pustulosa. Tetrahedron 1998; 54:13971-80.
[12] Holler U, Gloer J B, Wicklow D T. Biologically active polyketide metabolites from an undetermined fungicolous hyphomycete resembling cladosporium. J. Nat. Prod. 2002; 65:876-82.
[13] Jiang B, Smallheer J M, Amarally C, Wuonola M A. Total synthesis of (+/−)-dragmacidin—a cytotoxic bis(indole)alkaloid of marine origin. J. Org. Chem. 1994; 59:6823-7.
[14] Johnson A L, Bergman J, Sjögren M, Bohlin L. Synthesis of barettin. Tetrahedron 2004; 60:961-5.
[15] Johnson A L, Janosik T, Bergman J. Synthesis of the diketopiperazine dipodazine. Arkivoc 2002 (viii):57-61.
[16] Kawahara H, Isoai A, Shizuri Y. Molecular cloning of a putative serotonin receptor gene from barnacle, Balanus amphitrite. Gene 1997; 184:245-50.
[17] Lieberknecht A, Griesser H. Amino-acids and peptides. 64. What is the structure of barettin—novel synthesis of unsaturated diketopiperazines. Tetrahedron Lett. 1987; 28:4275-8.
[18] McKee T C, Cardellina J H, Riccio R, Dauria M V, Iorizzi M, Minale L, Moran R A Gulakowski R J, McMahon J B, Buckheit R W, Snader K M, Boyd M R. Hiv-inhibitory natural-products. 11. Comparative-studies of sulfated sterols from marine-invertebrates. J. Med. Chem. 1994; 37:793-7.
[19] Okino T, Yoshimura E, Hirota H, Fusetani N. Antifouling kalihinenes from the marine sponge Acanthella-cavernosa. Tetrahedron Lett. 1995; 36:8637-40.
[20] Paul V J, Puglisi M P. Chemical mediation of interactions among marine organisms. Nat. Prod. Rep. 2004; 21:189-209.
[21] Peroutka S J, Howell T A. The molecular evolution of g-protein-coupled receptors—focus on 5-hydroxytryptamine receptors. Neuropharmacology 1994; 33:319-24.
[22] Prasad C. Bioactive cyclic dipeptides. Peptides 1995; 16:151-64.
[23] Qi X, Bakht S, Leggett M, Maxwell C, Melton R, Osbourn A. A gene cluster for secondary metabolism in oat: Implications for the evolution of metabolic diversity in plants. Proc. Natl. Acad. Sci. U.S.A. 2004; 101:8233-8.
[24] Sahm U G, Olivier G W J, Pouton C W. Synthesis of 153n-6 analogues and structure-function analysis at murine melanocortin-1 (mc1) receptors. Peptides 1999; 20:387-94.
[25] Siemion I Z, Gawlowska M, Krajewski K, Strug I, Wieczorek Z. Analogues of rgdvy and grgd peptides inhibit mycobacterium Kansasii phagocytosis. Peptides 2003; 24:1109-15.
[26] Sjögren M, Dahlström M, Göransson U, Jonsson P R, Bohlin L. Recruitment in the field of balanus improvisus and Mytilus edulis in response to the antifouling cyclopeptides barettin and 8,9-dihydrobarettin from the marine sponge Geodia barretti. Biofouling 2004; 20:291-7.
[27] Sjögren M, Göransson U, Johnson A L, Dahlström M, Andersson R, Bergman J, Jonsson P R, Bohlin L. Antifouling activity of brominated cyclopeptides from the marine sponge Geodia barretti. J. Nat. Prod. 2004; 67:368-72.
[28] Sölter S, Dieckmann R, Blumenberg M, Francke W. Barettin, revisited? Tetrahedron Lett. 2002; 43:3385-6.
[29] Sörensen D, Larsen T O, Christophersen C, Nielsen P H, Anthoni U. Dipodazine, a diketopiperazine from Penicillium dipodomys. Phytochemistry 1999; 51:1181-3.
[30] Stone M J, Williams D H. On the evolution of functional secondary metabolites (natural-products). Mol. Microbiol. 1992; 6:29-34.
[31] Towle M J, Salvato K A, Budrow J, Wels B F, Kuznetsov G, Aalfs K K, Welsh S, Zheng W J, Seletsky B M, Palme M H, Habgood G J, Singer L A, DiPietro L V, Wang Y, Chen J J, Quincy D A, Davis A, Yoshimatsu K, Kishi Y, Yu M J, Littlefield B A. In vitro and in vivo anticancer activities of synthetic macrocyclic ketone analogues of halichondrin b. Cancer Res. 2001; 61:1013-21.
[32] Tsukamoto S, Kato H, Hirota H, Fusetani N. Ceratinamides a and b: New antifouling dibromotyrosine derivatives from the marine sponge Pseudoceratina purpurea. Tetrahedron 1996; 52:8181-6.
[33] Tulp M, Bohlin L. Functional versus chemical diversity: Is biodiversity important for drug discovery? Trends Pharmacol. Sci. 2002; 23:225-31.
[34] Walker R J, Brooks H L, HoldenDye L. Evolution and overview of classical transmitter molecules and their receptors. Parasitology 1996; 113:S3-S33.
[35] Williams D H, Stone M J, Hauck P R, Rahman S K. Why are secondary metabolites (natural-products) biosynthesized. J. Nat. Prod. 1989; 52:1189-208.
[36] Wu D, Xing G W, Poles M A, Horowitz A, Kinjo Y, Sullivan B, Bodmer-Narkevitch V, Plettenburg O, Kronenberg M, Tsuji M, Ho D D, Wong C H. Bacterial glycolipids and analogues as antigens for cd1d-restricted nkt cells. Proc. Natl. Acad. Sci. U.S.A. 2005; 102:1351-6.

1. A compound of the general formula 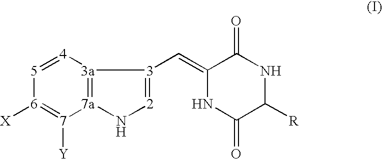
wherein
X and Y can be the same or different and can be any of H and R′, wherein
R′ is selected from —CH₃; —CH₂CH₃; —CH₃O; —Br; —Cl;
with the proviso that X and Y cannot both be —H; or
X and Y together form a ring structure selected from 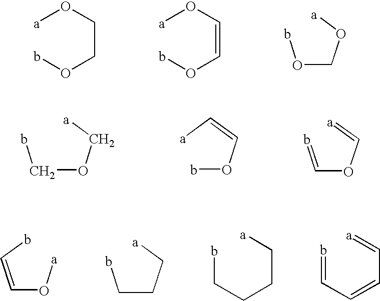
wherein a and b form points of attachment to positions 6 and 7, respectively, in formula (I);
and wherein
R is any of —H, —CH₃, —C₂H₅, C₃H₇, —CH═CH₂.

2. Benzo[g]dipodazine, according to the formula:

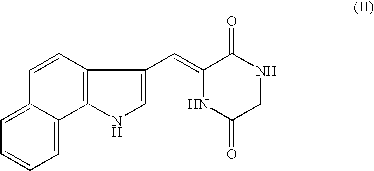

3. Surface on-growth inhibiting composition, comprising a compound according to any of claims 1 or 2 as an active agent.

4. Composition as claimed in claim 3, wherein the active agent is formulated with a suitable carrier.

5. Composition as claimed in claim 4, wherein the carrier is selected from vegetable oils, alkyd bases, acrylic bases.

6. A liquid linseed oil base composition comprising a compound according to any of claims 1 or 2 as an active agent.

7. A paste or lotion comprising a compound according to any of claims 1 or 2 as an active agent and a carrier selected from vegetable oils, preferably linseed oil, soy bean oil.

8. A film comprising a synthetic or natural polymer in which a compound according to claim 1 or 2 has been incorporated so as to be releasable.

9. A spray comprising a compound according to any of claims 1 or 2 as an active agent.

10. A method of inhibiting surface on-growth by organisms on various kinds of structures, comprising formulating active compound(s) according to claim 1 in a suitable carrier to form a coatable composition for application onto a surface to be protected against on-growth by organisms, such as cyprids and/or mussels in sea water, or bacteria in medical environments, e.g tubings and surgical equipment, and applying said composition onto a surface to be protected.

11. A method of inhibiting microfouling on the surface of surgical and other medical instruments or apparatuses, such as cutting tools for surgery, tubing for conveying body fluids such as blood, urine, by applying a composition as claimed in claim 3 to a surface of an object to be protected.
